(19) United States
(12) Patent Application Publication
(10) Pub. No.: US 2026/0118374 A1
(43) Pub. Date: Apr. 30, 2026

(54) IDENTIFICATION MARKERS FOR ACCURATELY DISTINGUISHING BETWEEN ANTEMORTEM ALCOHOL CONSUMPTION AND POSTMORTEM ALCOHOL INTRODUCTION, AND IDENTIFICATION METHOD USING IDENTIFICATION MARKERS

(71) Applicant: Shanxi Medical University, Taiyuan City (CN)

(72) Inventors: Keming YUN, Taiyuan City (CN); Lingxiao WANG, Taiyuan City (CN); Weichen LIU, Taiyuan City (CN); Xian JU, Taiyuan City (CN); Di LIANG, Taiyuan City (CN); Zixiang KAI, Taiyuan City (CN); Zhiwen WEI, Taiyuan City (CN); Chao ZHANG, Taiyuan City (CN)

(21) Appl. No.: 19/255,164

(22) Filed: Jun. 30, 2025

(30) Foreign Application Priority Data
Oct. 29, 2024 (CN) 202411516357.5

(51) Int. Cl. G01N 33/98

(52) U.S. Cl. CPC G01N 33/98

(57) ABSTRACT

Identification markers for accurately distinguishing between antemortem alcohol consumption and postmortem alcohol introduction, and an identification method using the identification markers are provided. The identification markers include: a concentration ratio of ethanol in a heart blood sample in a dead body to ethanol in a vitreous humor sample in the dead body, concentrations of ethanol in a muscle of a lower limb and a testis in the dead body, and concentrations of ethyl glucuronide (EtG) and ethyl sulfate (EtS) in the muscle of the lower limb and a testis in the dead body

### CROSS REFERENCE TO RELATED APPLICATION

This patent application claims the benefit and priority of Chinese Patent Application No. 202411516357.5 filed with the China National Intellectual Property Administration on Oct. 29, 2024, the disclosure of which is incorporated by reference herein in its entirety as part of the present application.

### TECHNICAL FIELD

The present disclosure belongs to the technical field of forensic identification, and specifically relates to identification markers for accurately distinguishing between antemortem alcohol consumption and postmortem alcohol introduction, and an identification method using the identification markers.

### BACKGROUND

In the process of forensic identification, determination of an origin of ethanol has always been a critical and challenging issue to be solved by forensic toxicologists. During decay of body fluids and tissues, nutrients could undergo fermentation under an action of microorganisms to produce ethanol^{[1]}. Moreover, an alcohol may be posthumously introduced into a body. Therefore, determining antemortem alcohol consumption solely based on a detection of ethanol is inaccurate. Therefore, identification of biomarkers for the ethanol consumed into the body before death, the ethanol produced in the body after death, and the ethanol introduced into the body after death is a key to explaining the origin of the ethanol.

Schmitt and Helander et al.^{[2-3]} have started a preliminary research on ethyl glucuronide (EtG) and ethyl sulfate (EtS) as biomarkers for ethanol. About 2% of the ethanol entering the body is metabolized through non-oxidative pathways^{[4]}. 0.5% of the ethanol is converted into the EtG under catalysis of an enzyme of uridine 5′-diphosphate-glucuronosyltransferase (UDP-UGT) superfamily with uridine 5′-diphosphate-glucuronic acid (UDP-GlcA) as a cofactor^{[5]}. 0.1% of the ethanol reacts with 3′-phosphoadenosine-5′-phosphosulfate under catalysis of a cytosolic sulfotransferase (SULT) to produce the EtS^{[6]}. There is a long detection window for the EtG and the EtS (a detection window of EtG in urine could be as long as 80 hours)^{[7-8]}. Recent evidence has also shown that, in in vitro blood, EtS exhibits high stability at 37° C., 25° C., 4° C., and −20° C. and EtG also exhibits high stability at low temperatures (4° C. and −20° C.). Further, after ethanol is added to in vitro blood and the in vitro blood is stored at 37° C. for 7 days, the EtG is produced^{[9]}. Previous studies on EtG and EtS production mostly rely on blood samples from subjects after drinking or blood samples produced by adding an ethanol standard to blank blood. However, in actual cases, there may be a situation where the alcohol is introduced into a deceased person to fabricate an evidence of antemortem alcohol consumption.

### SUMMARY

In order to address the problem that determination of an origin of ethanol in the current forensic identification is prone to false positives, the present disclosure provides identification markers for accurately distinguishing between antemortem alcohol consumption and postmortem alcohol introduction, and an identification method using the identification markers.

The present disclosure is achieved through the following technical solutions.

Identification markers for accurately distinguishing between antemortem alcohol consumption and postmortem alcohol introduction includes: a concentration ratio of ethanol in a heart blood to ethanol in a vitreous humor, ethanol in a muscle of a lower limb and a testis, and EtG and EtS in the muscle of the lower limb and the testis.

The identification markers include: a concentration ratio of ethanol in a heart blood sample in a dead body to ethanol in a vitreous humor sample in the dead body, ethanol in a muscle of a lower limb and a testis in the dead body, and EtG and EtS in the muscle of the lower limb and the testis in the dead body.

A method for accurately distinguishing between the antemortem alcohol consumption and the postmortem alcohol introduction using the identification markers, where
if the concentration ratio of the ethanol in the heart blood sample to the ethanol in the vitreous humor sample is less than 1 in the dead body, antemortem alcohol consumption is determined for the dead body;
if the concentration ratio of the ethanol in the heart blood sample to the ethanol in the vitreous humor sample is more than or equal to 1 and the ethanol shows positive in the muscle of the lower limb and the testis in the dead body, the antemortem alcohol consumption is determined for the dead body;
if the ethanol shows negative in the muscle of the lower limb and the testis and the EtG and the EtS show positive in the muscle of the lower limb and the testis in the dead body, the antemortem alcohol consumption is determined for the dead body; and
if the concentration ratio of the ethanol in the heart blood sample to the ethanol in the vitreous humor sample is more than or equal to 1, the ethanol shows negative in the muscle of the lower limb and the testis, and the EtG and the EtS show negative in the muscle of the lower limb and the testis in the dead body, the postmortem alcohol introduction is determined for the dead body.

In the present disclosure, ethanol is infused into rabbit carcasses, and then diffusion of the ethanol and generation of EtG and EtS in the rabbit carcasses are detected, so as to provide experimental evidence for explanation of an origin of the ethanol in a forensic case investigation process. Results show that, at high temperatures, infusion of the ethanol into rabbits shortly after death will cause the generation of the EtG and the EtS in body fluids and tissues, which poses a new challenge to the determination of the origin of the ethanol. Therefore, in a forensic case investigation process, determining whether there is drinking should involve the detection of ethanol concentrations in heart blood and vitreous humor samples, ethanol in a muscle of a lower limb and a testis, and EtG and EtS in the muscle of the lower limb and the testis.

According to experimental results of the present disclosure, in an actual case investigation process, if a concentration ratio of the ethanol in a heart blood to the ethanol in a vitreous humor is far more than 1, it indicates that the ethanol may be introduced after death. In addition to the concentration ratio of the ethanol in the heart blood, ethanol, EtG and EtS in the muscle of the lower limb and the testis should also be detected to assist in determining whether there is drinking.

### BRIEF DESCRIPTION OF THE DRAWINGS

FIG. 1 shows a chromatogram for EtG and EtS in a rabbit blood;

FIG. 2 shows a chromatogram for the EtG and the EtS in a rabbit liver;

FIG. 3 shows concentrations of ethanol in body fluids and tissues resulting from intragastric administration of ethanol at different time points after death according to an embodiment of the present disclosure;

FIG. 4 shows concentrations of EtG in body fluids and tissues resulting from intragastric administration of ethanol at different time points after death according to an embodiment of the present disclosure; and

FIG. 5 shows concentrations of EtS in body fluids and tissues resulting from intragastric administration of ethanol at different time points after death according to an embodiment of the present disclosure.

### DETAILED DESCRIPTION OF THE EMBODIMENTS

To make objectives, technical solutions, and advantages of embodiments of the present disclosure clear, the technical solutions in the embodiments of the present disclosure will be described clearly and completely below. Obviously, the embodiments described are merely some rather than all of the embodiments of the present disclosure. All other embodiments obtained by those of ordinary skill in the art based on the embodiments of the present disclosure without creative efforts shall fall within the scope of the present disclosure.

In some embodiments, the postmortem alcohol introduction is conducted by intragastric administration.

Unless otherwise specified, all technical and scientific terms used herein have the same meanings as commonly understood by those skilled in the art to which the present disclosure belongs.

All public references and materials cited thereby will be incorporated by reference.

Equivalent technologies of specific embodiments described that are recognized by those skilled in the art and could be understood through conventional experiments will be included in the present application.

Unless otherwise specified, all experimental methods in the following embodiments are conventional methods. All instruments adopted in the following embodiments are conventional laboratory instruments, unless otherwise specified. All experimental materials adopted in the following embodiments are purchased from conventional biochemical reagent stores, unless otherwise specified.

In order to clarify an origin of ethanol in a dead body, ethanol was infused into rabbit carcasses, and concentrations of ethanol and metabolites EtG and EtS thereof in body fluids and tissues were detected.

### 1. Animal Experiment:

15 male New Zealand white rabbits each with a body weight of 2.0 kg±0.2 kg (Experimental Animal Center of Shanxi Medical University) were selected and fasted overnight before the experiment. The male New Zealand white rabbits each were fixed on an operating table, a gastric tube was inserted, and a trachea was clamped until death (expiration and heartbeat were stopped). Then resulting rabbit carcasses were divided into 5 groups (n=3). At 37° C., the resulting rabbit carcasses were subjected to intragastric administration with ethanol at a dose of LD₅₀ (LD₅₀=6.3 g/kg) immediately after the death and at 1 hour, 2 hours, 4 hours, and 8 hours after the death. 24 hours after the intragastric administration, anatomy was conducted, and a heart blood, an inferior vena cava blood, a vitreous humor, a liver, a lung, a kidney, a brain, a spleen, a muscle of a left lower limb, and a testis were collected and tested for EtG and EtS.

### 2. Main Reagents and Instruments:

Methanol at a chromatographic grade and acetonitrile at a chromatographic grade (Sigma-Aldrich, the United States); formic acid at a chromatographic grade (Aladdin, Shanghai, China); tert-butanol and ethanol standards (Academy of Forensic Science, Shanghai, China); 25% ammonium hydroxide (Damao, Tianjin, China); nitrogen (Anxu Hongyun Technology, Shanxi, China); Bond-Elut NH₂ solid-phase extraction cartridge (Agilent, the United States); EtG (Sigma-Aldrich, the United States); deuterated ethyl glucuronide (D5-EtG, IS) (Sigma-Aldrich, the United States); deuterated ethyl sulfate (D5-EtS, IS) (Sigma-Aldrich, the United States); EtS (Sigma-Aldrich, the United States); and ultrapure water adopted each time is freshly produced by a water purifier.

Agilent 1260 liquid chromatography-Agilent 6460 Triple Quadrupole tandem mass spectrometry (Agilent, the United States); headspace sampler-gas chromatography system (Agilent, the United States); Pressure+48 solid-phase extraction instrument (Biotage, Switzerland); Votex-genie 2 vortex mixer (Scientific Industries, the United States); Euro automatic nitrogen purge instrument (Eurotherm Ltd., Shanghai, China); Milli-Q water purifier (Millipore, the United States); pipettes (1 μL to 10 μL, 10 μL to 100 μL, and 100 μL to 1,000 μL, Eppendorf, Germany); hydrogen/air integrated generator HA-500 (Beijing BCHP Analytical Technology Institute, Beijing, China); and constant climate chamber (Ningbo Yanghui Instruments Co., Ltd., Ningbo, China).

### 3. Sample Treatment

(1) Ethanol extraction: According to a method described in a reference [10]. For a calibration sample, a quality control sample, and other samples for methodological validation, 100 μL of an ethanol working solution at a specified concentration and 500 μL of a tert-butanol internal standard working solution (40 μg/mL) each were taken and added to a sample vial, the sample vial was sealed with an aluminum cap carrying a silicone rubber gasket by sealing pliers, and thorough mixing was conducted to get ready for injection. For a body fluid sample to be tested, 100 μL of a body fluid was taken and added to a sample vial. For a tissue sample, 1 μg of a homogenized tissue to be tested was taken, 2 mL of deionized water was added, vortex mixing was conducted for 5 minutes, centrifugation was conducted at 3,500 rpm for 5 minutes, and 100 μL of a resulting supernatant was taken and added to a sample vial. The rest was the same as for the calibration sample. Analysis was conducted with a headspace-gas chromatography system (HS-GC).

(2) Extraction of EtG and EtS: According to an extraction method established in references [10,11]: 100 μL of a whole blood was taken and added to a 1.5 mL centrifuge tube. 10 μL of a mixed internal standard working solution (D₅-EtG and D₅-EtS, each 2.5 μg/mL) and 400 μL of deionized water were added in sequence. Vortexing was conducted for 30 seconds to allow thorough mixing for loading.

Method 1: A solid-phase extraction cartridge was activated with 1.2 mL of a methanol (0.3% of formic acid) solution and 1.2 mL of deionized water in sequence, and a nitrogen pressure was adjusted to 2 Psi to 3 Psi. A sample was loaded. When the sample was fully dispersed in a packing, 1.2 mL of the methanol (0.3% of the formic acid) solution was added for acidification, which was conducted at a flow rate of 1 drop/2 seconds. Drying was conducted for 10 minutes under a nitrogen flow of 5 Psi. Then elution was conducted with 1.2 mL of a methanol-water solution of 5% NH₄OH at the same flow rate. An eluate was dried under a nitrogen flow in a 35° C. water bath. A resulting solid was re-dissolved with 200 μL of 10% acetonitrile, then filtered through a filter membrane, and then analyzed by high-performance liquid chromatography-tandem mass spectrometry (LC-MS/MS).

Method 2: 100 mg of the homogenized tissue to be tested was taken, and 20 μL of a mixed internal standard working solution (D₅-EtG and D₅-EtS, each 25 μg/mL), 100 μL of deionized water, and 800 μL of acetonitrile were added. Shaking was conducted for 5 minutes to allow thorough mixing, and an ultrasonic treatment was conducted in an ice bath for 30 minutes. Centrifugation was conducted for 10 minutes at 13,000 rpm and 4° C. A resulting supernatant was taken, then dried under a nitrogen flow in a 35° C. water bath. An obtained solid was re-dissolved with 200 μL of the 10% acetonitrile, then filtered through a filter membrane, and then analyzed by the LC-MS/MS.

### (3) Instrument Conditions

HS-GC: According to the national standard (GAT1073-2013), parameters of a headspace automatic sampler were set as follows: a temperature of a heating chamber was 65° C.; a temperature of a sample loop was 105° C., a temperature of a transmission line was 110° C.; a circulation time of a gas phase was 3.5 minutes; a heat equilibrium time of a sample vial was 10.0 minutes; a pressurization time of the sample vial was 0.10 minutes; a filling time of the sample loop was 0.10 minutes; an equilibration time of the sample loop was 0.05 minutes; and an injection time was 1.0 minutes.

A gas chromatography column was an Agilent DB-ALC1 (30 m*0.32 mm*1.8 m) column, with a constant temperature of 40° C. A flow rate was 8 mL/minute. A temperature of an injection port was 150° C. In a split mode, a split ratio was 10:1. A temperature of a flame ionization detector (FID) was 250° C. A H₂ flow rate was 30 mL/minute. An air flow rate was 360 mL/minute. A make-up gas flow rate of N₂ was 25 mL/minute.

### LC-MS/MS Conditions:

A chromatographic column was Agilent Zorbax SB-C18 (2.1*100 mm; 3.5-Micron). A mobile phase was water-acetonitrile (90:10). A flow rate was 0.4 mL/minutes. An injection volume was 5 μL. A column temperature was 25° C. A duration of single analysis was 4.0 minutes.

An ionization mode was electrospray ionization (ESI). An operating mode was multiple reaction monitoring (MRM). Mass spectrometry parameters for each target compound are shown in Table 1.

TABLE 1
MRM parameters for each target compound

| | Precursor | | Daughter | Collision |
|---|---|---|---|---|
| Compound | ion (m/z) | Fragmentor | ion (m/z) | energy (eV) |
| EtG | 221 | 105 | 85 | 12 |
| | | | 75* | 14 |
| D₅-EtG | 226 | 100 | 85 | 15 |
| | | | 75* | 16 |
| EtS | 125 | 90 | 97* | 14 |
| | | | 80 | 35 |
| D₅-EtS | 130 | 95 | 98* | 21 |
| | | | 80 | 46 |

Note:
*indicates a quantitative ion.

### 4. Methodological Validation:

The methodological validation was carried out according to industry guidelines[¹²], including specificity, limit of detection (LOD), lower limit of quantitation (LLOQ), linearity, precision and accuracy, recovery rate, matrix effect (ME), and stability.

(1) Specificity: 10 blank rabbit blood samples and 10 blank rabbit liver samples were collected from different sources. EtG and EtS each were added at a dose of 20% LLOQ. Each sample was treated by the above sample treatment method and then analyzed by LC-MS/MS for specificity investigation.

(2) LOD and LLOQ: A concentration at which a signal-to-noise ratio (SNR) of an analyte in a sample was larger than or equal to 3 was defined as the LOD. A minimum concentration at which the SNR of the analyte in the sample was larger than or equal to 10 was defined as the LLOQ.

(3) Linearity: 100 μL of the blank rabbit blood sample and 1 μg of the blank rabbit liver sample were taken, and an ethanol standard was added to prepare blood and tissue samples for a working curve in a concentration range of 5 mg/100 mL to 800 mg/100 mL. Each sample was treated by the above sample treatment method, and then analyzed by HS-GC.

EtG and EtS standard solutions each were added to 100 μL of the blank rabbit blood samples to prepare blood samples for working curves in concentration ranges of 25 ng/mL to 2,500 ng/mL and 40 ng/mL to 2,500 ng/mL, respectively. The EtG and EtS standard solutions each were added to 100 mg of the blank rabbit liver sample to prepare tissue samples for a working curve in a concentration range of 0.05 ng/mg to 20 ng/mg. Each sample was treated by the sample treatment method in methods 1-2, and then analyzed by LC-MS/MS.

(4) Precision and accuracy: An ethanol standard sample was added to each of the blank rabbit blood samples and the blank rabbit liver samples to prepare four concentrations of LLOQ (5 mg/100 mL), L (10 mg/100 mL), M (200 mg/100 mL), and H (800 mg/100 mL). Each sample was treated as above and then analyzed. 6 replicate samples were set for each concentration, and there were 24 samples in total for each batch. 5 batches were analyzed in total. 1st, 2nd, and 3rd batches were detected in the morning, at noon, and in the afternoon of the first day to calculate an intra-day accuracy and precision. 4th and 5th batches were detected at noon on day 2 and day 3 to calculate an inter-day accuracy and precision. The accuracy and the precision were calculated according to a working curve.

EtG and EtS standards each were added to the blank rabbit blood samples to prepare the four concentrations of LLOQ (25 ng/mL and 40 ng/mL), L (100 ng/mL), M (1,000 ng/mL), and H (2,500 ng/mL). The EtG and EtS standards each were added to the blank rabbit liver samples to prepare four concentrations of LLOQ (0.05 ng/mg), L (0.1 ng/mg), M (1 ng/mg), and H (20 ng/mg). Each sample was treated according to methods 1-2 and then analyzed. The precision and the accuracy were calculated as above.

(5) Recovery rate and ME: Recovery rates of EtG and EtS in blood and liver samples during an extraction process were determined by comparing an analyte concentration in a sample with a standard added before extraction with analyte concentrations in samples with standards at the three concentrations (L, M, and H) added after extraction (n=6).

Blank blood and liver samples were extracted, and the EtG and EtS standards were added to evaluate ME (n=6). A peak area (A) of EtG or EtS in a sample of a matrix+a standard at the L, M, and H concentrations was compared with a peak area (B) of EtG or EtS in a sample of water+the standard at the same concentrations, respectively. The ME was defined as a percentage ratio of A to B. The ME of 100% indicated no ME. The ME of higher than 100% indicated enhancement of ionization. The ME of lower than 100% indicated inhibition of ionization.

(6) Stability: Sample preparation was the same as for the accuracy and the precision. There were three batches of samples in total. A first batch was treated by the above sample treatment method, and then analyzed. A second batch was subjected to 3 freeze-thaw cycles (−20° C. to room temperature), then treated by the above sample treatment method, and then analyzed. A third batch was treated by the above sample treatment method, then placed in a sampler for 24 hours, and then analyzed. The stability during repeated freeze-thaw cycles and the stability in the sampler were expressed by a deviation: deviation=(actual concentration−measured concentration)/actual concentration.

### 5. Experimental Results

### (1) Results of the Methodological Validation

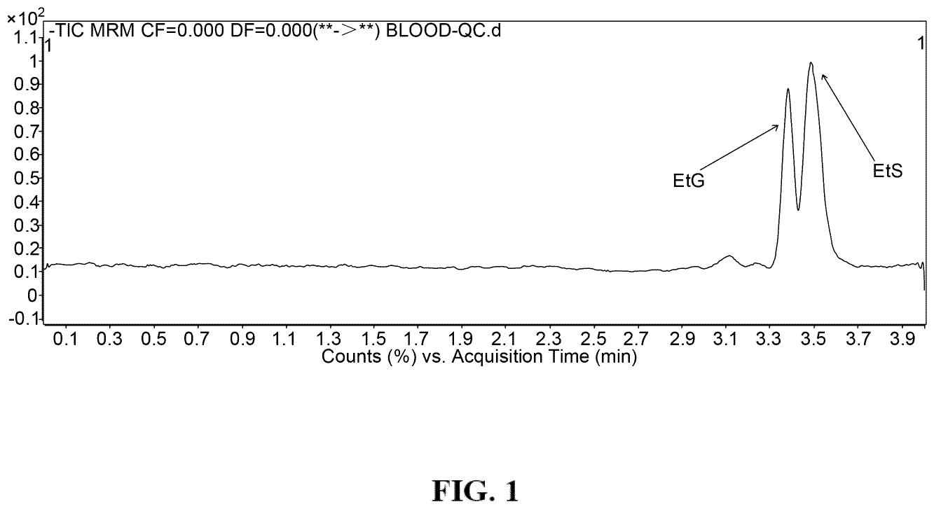

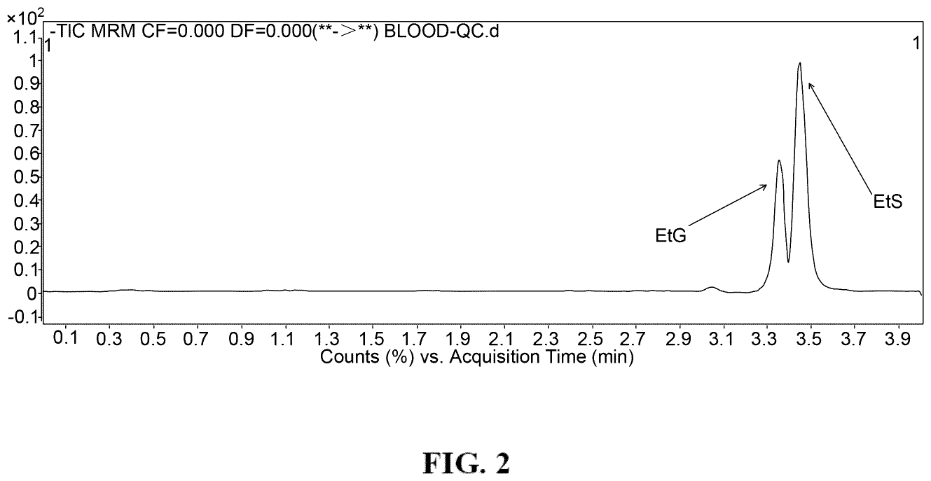

A. Specificity: EtG and EtS detection method in this experiment exhibited strong specificity, enabled a prominent separation effect for target substances, and was not interfered with by matrices in blood and tissue samples, as shown in FIG. 1 and FIG. 2.

B. LOD, LLOQ, and linearity: Ethanol showed an excellent linear relationship in a range of 5 mg/100 mL to 800 mg/100 mL, and correlation coefficients R² all were greater than 0.9999. A sample with a too-high ethanol concentration should be first diluted. A final result was a measured concentration*a dilution factor. EtG and EtS in blood samples showed prominent linear relationships in ranges of 25 ng/mL to 2,500 ng/mL and 40 ng/mL to 2,500 ng/mL, and correlation coefficients R² all were greater than 0.999. EtG and EtS in liver samples showed prominent linear relationships in a range of 0.05 μg/mg to 20 ng/mg, and correlation coefficients R² all were greater than 0.99. The LOD, the LLOQ, and linear equations are shown in Table 2.

TABLE 2
LOD, LLOQ, and linearity for ethanol, EtG, and EtS

| | LOD | LLOQ | |
|---|---|---|---|
| Compound | (ng/mL) | (ng/ml) | Linear equation |
| EtG | 20²⁾ | 25²⁾ | y = 0.004893*x + 0.08884 |
| | 0.01³⁾ | 0.05³⁾ | y = 0.007113*x + 0.01681 |
| EtS | 25²⁾ | 40²⁾ | y = 0.005482*x + 0.10958 |
| | 0.01³⁾ | 0.05³⁾ | y = 0.002843*x + 0.03842 |
| Ethanol¹⁾ | 2 | 5 | y = 0.048910*x + 0.06020 |

Notes:
¹⁾indicates an ethanol unit of mg/100 mL.
²⁾indicates a blood result.
³⁾indicates a liver result.

C. Precision, accuracy, recovery rate, ME, and stability: Recovery rate, ME, and stability results are shown in Table 3. An inter-day precision and an intra-day precision both were in a range of 1.5000 to 6.3700, and an inter-day accuracy and an intra-day accuracy both were in a range of 86.72% to 104.33%, which met industry requirements.

TABLE 3
Recovery rate, ME, and stability for EtG and EtS (n ± 6, x ± s)

| | | | | Stability change rate (%) | |
|---|---|---|---|---|---|
| | | Recovery | | During repeated | |
| | | rate | | freeze-thaw | In a |
| Compound | Concentration | (%) | ME (%) | cycles | sampler |
| EtG | L | 89.2 ± 2.3¹⁾ | 86.1 ± 4.2¹⁾ | 5.2 ± 0.3¹⁾ | 6.3 ± 0.5¹⁾ |
| | | 87.4 ± 8.5²⁾ | 89.2 ± 9.5²⁾ | 4.3 ± 1.1²⁾ | 5.2 ± 0.7²⁾ |
| | M | 95.3 ± 5.1¹⁾ | 88.2 ± 8.4¹⁾ | 2.8 ± 0.2¹⁾ | 3.4 ± 0.8¹⁾ |
| | | 90.2 ± 7.1²⁾ | 84.9 ± 7.2²⁾ | 7.7 ± 0.5²⁾ | 4.1 ± 0.6²⁾ |
| | H | 101 ± 8.4¹⁾ | 79.2 ± 7.1¹⁾ | 3.5 ± 0.4¹⁾ | 5.1 ± 0.4¹⁾ |
| | | 88 ± 2.3²⁾ | 85.5 ± 6.9²⁾ | 4.2 ± 0.6²⁾ | 6.7 ± 0.1²⁾ |
| EtS | L | 88.5 ± 7.3¹⁾ | 89.1 ± 4.5¹⁾ | 3.2 ± 0.2¹⁾ | 3.0 ± 0.3¹⁾ |
| | | 79.2 ± 5.5²⁾ | 82.3 ± 3.1²⁾ | 1.2 ± 0.1²⁾ | 4.4 ± 1.0²⁾ |
| | M | 99.4 ± 3.6¹⁾ | 85.9 ± 8.8¹⁾ | 5.7 ± 0.4¹⁾ | 2.5 ± 0.7¹⁾ |
| | | 81.2 ± 3.4²⁾ | 84.2 ± 8.4²⁾ | 3.3 ± 0.3²⁾ | 6.3 ± 0.8²⁾ |
| | H | 96.5 ± 3.2¹⁾ | 89.7 ± 9.3¹⁾ | 6.2 ± 0.1¹⁾ | 3.2 ± 1.0¹⁾ |
| | | 80.2 ± 6.7²⁾ | 90.6 ± 4.1²⁾ | 4.6 ± 0.6²⁾ | 3.6 ± 1.0²⁾ |

Notes:
¹⁾indicates a blood result; and
²⁾indicates a liver result.

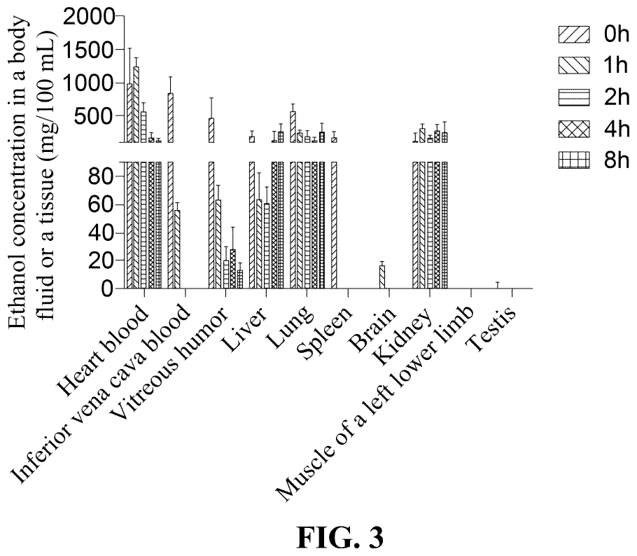

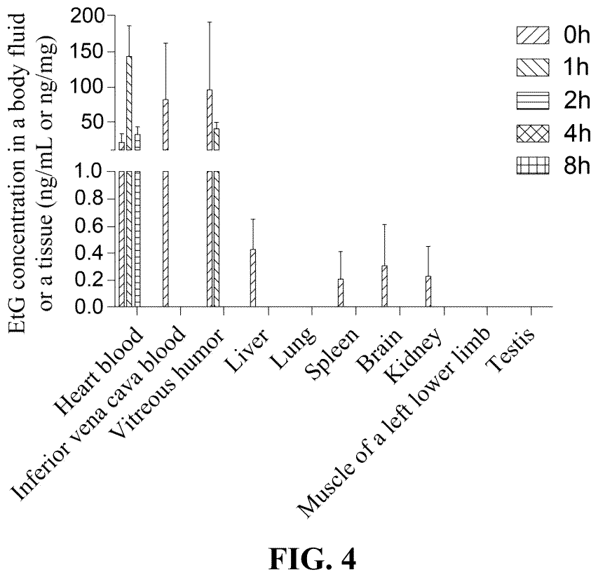

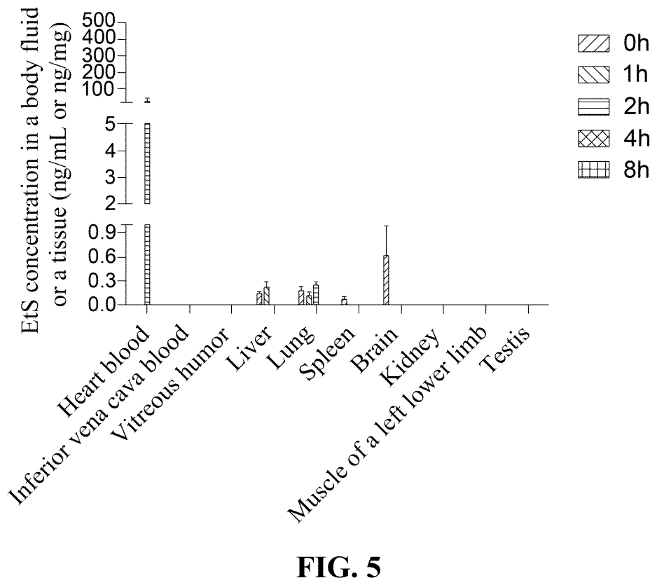

(2) Results of the animal experiment: At 0 hour, 1 hours, 2 hours, 4 hours, and 8 hours after death, the rabbit carcasses each were subjected to intragastric administration with ethanol at LD₅₀ and then kept at 37° C. for 24 hours. Concentrations of ethanol, EtG, and EtS in body fluids and organs were then detected. According to results, diffusion of the ethanol occurred in all rabbit carcasses, a diffusion degree of the ethanol in each body fluid (a heart blood, an inferior vena cava blood, and a vitreous humor) decreased with extension of an intragastric administration time point after death, and there was a consistent diffusion degree in the organs. No ethanol was detected in a muscle of a left lower limb and a testis in each of 5 postmortem intragastric administration groups, ethanol was detected in spleens of the postmortem intragastric administration groups quickly at 0 hour after the death, and the ethanol was detected in brains of the postmortem intragastric administration groups quickly at 1 hours after the death, as shown in FIG. 3. When the intragastric administration was conducted long after the death (at 4 hours and 8 hours), neither EtG nor EtS was detected in all tested body fluids and tissues. However, when the intragastric administration was conducted shortly after death (at 0 hour, 1 hours, and 2 hours), the EtG and the EtS both were detected in the heart blood, a liver, the spleen, and the brain, only the EtG was detected in the inferior vena cava blood, the vitreous humor, and a kidney, and only the EtS was detected in a lung. Specific concentrations are shown in Tables 4 and 5. Neither the EtG nor the EtS was detected in the muscle of the left lower limb and the testis at any of 5 postmortem intragastric administration time points, as shown in FIG. 4 and FIG. 5. In addition, in a blank group, the ethanol, the EtG, and the EtS were not detected in all body fluids or tissues.

TABLE 4
Ethanol concentrations (mg/100 mL) in various body
fluids and tissues of rabbits at different time
points of postmortem intragastric administration

| Body fluid or tissue | 0 hour | 1 hours | 2 hours | 4 hours | 8 hours |
|---|---|---|---|---|---|
| Heart blood | 970.54 | 1242.36 | 549.69 | 165.86 | 121.35 |
| Inferior vena | 836.32 | 55.89 | — | — | — |
| cava blood | | | | | |
| Vitreous humor | 458.90 | 63.42 | 19.96 | 27.30 | 12.55 |
| Liver | 177.73 | 63.98 | 60.69 | 130.64 | 263.26 |
| Lung | 567.07 | 248.95 | — | 113.80 | 247.52 |
| Spleen | 165.22 | — | — | — | — |
| Brain | — | 16.02 | — | — | — |
| Kidney | 116.36 | 311.77 | 171.08 | 266.19 | 241.60 |
| Muscle of a | — | — | — | — | — |
| left lower limb | | | | | |
| Testis | — | — | — | — | — |

Note:
— indicates that a concentration is lower than LOD.

TABLE 5
Concentrations of EtG and EtS in body fluids and tissues at different
time points of postmortem intragastric administration

| | | 0 | 1 | 2 | 4 | 8 |
|---|---|---|---|---|---|---|
| Body fluid or tissue | | hour | hours | hours | hours | hours |
| Heart blood¹⁾ | EtG | 21.14 | 143.10 | 32.43 | — | — |
| | EtS | — | — | 17.06 | — | — |
| Inferior vena cava | EtG | 80.61 | — | — | — | — |
| blood¹⁾ | EtS | 105.66 | — | — | — | — |
| Vitreous humor¹⁾ | EtG | 95.19 | 40.17 | — | — | — |
| | EtS | 232.50 | — | — | — | — |
| Liver²⁾ | EtG | 0.43 | — | — | — | — |
| | EtS | 0.13 | 0.22 | — | — | — |
| Lung²⁾ | EtG | — | — | — | — | — |
| | EtS | 0.17 | 0.12 | 0.24 | — | — |
| Spleen²⁾ | EtG | 0.20 | — | — | — | — |
| | EtS | 0.06 | — | — | — | — |
| Brain²⁾ | EtG | 0.30 | — | — | — | — |
| | EtS | 0.45 | — | — | — | — |
| Kidney²⁾ | EtG | 0.22 | — | — | — | — |
| | EtS | — | — | — | — | — |
| Muscle of a left | EtG | — | — | — | — | — |
| lower limb²⁾ | EtS | — | — | — | — | — |
| Testis²⁾ | EtG | — | — | — | — | — |
| | EtS | — | — | — | — | — |

Notes:
— indicates that a concentration is lower than LOD.
¹⁾indicates a concentration unit of ng/mL.
²⁾indicates a concentration unit of ng/mg.

EtG and EtS, as non-oxidative metabolites of ethanol, are widely used for distinguishing between antemortem alcohol consumption and postmortem ethanol production. However, some studies have shown that the addition of ethanol to in vitro blood at 37° C. could cause generation of the EtG^{[9]}. In an actual case investigation process, there is a situation where a dead body is introduced with an alcohol to fake a scene. Currently, there are no clear identification indexes and methods to distinguish between antemortem ethanol consumption and the postmortem ethanol production and to determine whether the EtG and the EtS will be generated after postmortem alcohol introduction.

In the present disclosure, situations where an alcohol is introduced under a high-temperature condition at different time points after death are simulated with rabbits, and the ethanol, the EtG, and the EtS in the bodily fluids and the organs are detected. Results show that a concentration ratio of the ethanol in the heart blood to the ethanol in the vitreous humor is 13.00±10.40. However, according to a study on redistribution of ethanol in dogs after death in the existing literature, a concentration ratio of ethanol in a heart blood to ethanol in a vitreous humor at 24 hours after death is 0.94±0.03^{[13]} (less than 1). These results show that the concentration ratio of the ethanol in the heart blood to the ethanol in the vitreous humor in the case of postmortem ethanol introduction is greater than the concentration ratio of the ethanol in the heart blood to the ethanol in the vitreous humor in the case of the antemortem alcohol consumption. This is because the heart is closer to a stomach than the vitreous body and the heart blood is more easily affected by postmortem diffusion than the vitreous humor. Therefore, when the antemortem alcohol consumption is distinguished from the postmortem alcohol introduction, the concentration ratio of the ethanol in the heart blood to the ethanol in the vitreous humor could be referenced. In the blank group of this experiment, the ethanol, the EtG, and the EtS were not detected. Thus, influence of the postmortem ethanol production on a research result is ruled out.

It can be seen from Table 5 that, when the ethanol is introduced within a short period after death (0 hour to 2 hours), the EtG or the EtS could be detected in all body fluids and organs except for the muscle of the left lower limb and the testis. Vitreous humors, as an alternative specimen for blood, are less affected by microorganisms and postmortem redistribution^{[14-15]}. Thus, the vitreous humors are widely used in identification of poisons. In this experiment, when rabbits were subjected to the intragastric administration with ethanol immediately after death, the ethanol, the EtG, and the EtS all were detected in the vitreous humor with concentrations of 458.90 mg/100 mL, 95.19 ng/mL, and 232.50 ng/mL, respectively. When the intragastric administration was conducted at 1 hour after death, the ethanol and the EtG were detected in the vitreous humor with concentrations of 63.42 mg/100 mL and 40.17 ng/mL, respectively. As a result, determining whether there was the antemortem alcohol consumption solely based on the detection of the EtG and the EtS in the vitreous humor is at a risk of false positives. Whether there was the antemortem alcohol consumption could be comprehensively determined in combination with concentrations of the EtG and the EtS in the tissues such as the muscle of the lower limb and the testis that are far from the heart and the stomach.

Ethanol reacts with glucuronides and sulfates under catalysis of glucuronyl transferases (UGTs) and sulfotransferases (SULTs) in vivo to produce EtG and EtS, respectively. When a body dies, enzymes in the body are not inactivated immediately. In this case, a part of ethanol could still be metabolized under catalysis of the enzymes^{[1]}. This experiment was carried out at a high temperature, and rabbits were placed at 37° C. immediately after being sacrificed. Some enzymes in carcasses still remain active. As a result, the EtG and the EtS were generated in the body fluids and the organs when the ethanol was intragastrically administered shortly after death. There are reports in the literature that UGTs and SULTs also exist in Escherichia coli^{[21]}. At high temperatures, corpses decompose rapidly, and microorganisms may cause production of the EtG and the EtS from ethanol under an action of the UGTs and the SULTs in the microorganisms. UGTs and SULTs show tissue specificity in vivo^{[16-17]}. Enzymes for producing EtG are mainly two subtypes of UGT1A1 and UGT2B7^{[5]}. mRNA of UGTIA1 is observed in a liver and a small intestine, but is not observed in a colon, a kidney, and a brain^{[18]}. Enzymes for producing EtS are mainly SULT1A1^{[19]}. The research by Riley et al. indicates that SULTIA1 is primarily expressed in livers, small intestines, and large intestines of rabbits^{[20]}. As shown in FIG. 2 and FIG. 3 of the present disclosure, the EtS is mainly produced in the organs, while the EtG is primarily produced in the body fluids, which also confirms the above research. Based on a comprehensive analysis of the above results, the production of the EtG and the EtS in the body fluids and the tissues after the intragastric administration of the ethanol to the rabbit carcasses may be caused by a combined action of enzymes not inactivated themselves and microorganisms resulting from decaying.

In the actual case investigation process, if the concentration ratio of the ethanol in the heart blood to the ethanol in the vitreous humor is far more than 1, it indicates that ethanol may be introduced after death. In addition to the EtG and the EtS in the heart blood, the ethanol in the muscle of the lower limb and the testis should also be detected to assist in determining whether there is drinking. Due to the small size of the rabbits and the significant diffusion effect of the ethanol in the rabbits, large animals such as miniature pigs or monkeys will be adopted in the subsequent experiments for simulation to observe production of EtG and EtS in body fluids and tissues.

Finally, it should be noted that the above embodiments are merely intended to describe rather than limit technical solutions of the present disclosure. Although the present disclosure is described in detail with reference to the above embodiments, those of ordinary skill in the art should understand that modifications may be made to the technical solutions described in the above embodiments or equivalent replacements may be made to some or all technical features thereof, which do not make the essence of the corresponding technical solutions depart from the scope of the technical solutions in the embodiments of the present disclosure.

### CITED REFERENCES

[1] Sastre C, Bartoli C, Baillif-Couniou V, et al. Post Mortem Redistribution of Drugs: Current State of Knowledge. Curr Pharm Des. 2017; 23(36):5530-5541. doi: 10.2174/1381612823666170622111739.
[2] Schmitt G, Aderjan R, Keller T, et al. Ethyl glucuronide: an unusual ethanol metabolite in humans. Synthesis, analytical data, and determination in serum and urine[J]. J Anal Toxicol. 1995, 19(2):91-94. doi:10.1093/jat/19.2.91.
[3] Helander A, Beck O. Mass spectrometric identification of ethylsulfate as an ethanol metabolite in humans[J]. Clin Chem. 2004, 50(5):936-7.doi: 10.1373/clinchem.2004.031252.
[4] Liu Liang and Zhang Guohua. Forensic Toxicology[M]. 5th ed. Beijing: People's Medical Publishing House, 2014: 164-166.
[5] Liu L, Zhang G H. Forensic toxicology[M]0.5th ed. Beijing: People's Medical Publishing House, 2014:164-166.
[6] Foti R S, Fisher M B. Assessment of UDP-glucuronosyltransferasecatalyzed formation of ethyl glucuronide in human liver microsomes and recombinant UGTs[J]. Forensic Sci Int. 2005, 153(2-3):109-16. doi: 10.1016/j.forsciint.2004.12.003.
[7] Hugbart C, Verres Y, Le Dare B, et al. Non-oxidative ethanolmetabolism in human hepatic cells in vitro: Involvement of uridine diphospho-glucuronosyltransferase 1A9 in ethylglucuronide production[J]. Toxicol InVitro. 2020, 66:104842. doi:10.1016/j.tiv.2020.104842.
[8] Zhao Hui. Research on Detection and Kinetics of Biomarker Metabolites of Ethanol[D]. Shanghai: Fudan University, 2010.
[9] Zhao H. Studies on detection of Minor Metabolites of ethano and ethyl glucuronide metabolism in Chinese population[D]. Shanghai:ShanghaiMedical College Fudan University, 2010.
[10] Ren Lei. Detection of Ethyl Sulfate (EtS) in Blood and Urine by High-Performance Liquid Chromatography-Tandem Mass Spectrometry and Use thereof in Forensic Medicine[D]. Chongqing: Chongqing Medical University, 2013.
[11] Ren L. Determination of ethyl sulfate in serum and urine by HPLC-MS/MS and its application in forensic[D]. Chongqing:Congqing Medical University, 2013.
[12] Liu Y, Zhang X, Li J, et al. Stability of Ethyl Glucuronide, Ethyl Sulfate, Phosphatidylethanols and Fatty Acid Ethyl Esters in Postmortem Human Blood[J]. J Anal Toxicol. 2018; 42(5):346-352. doi:10.1093/jat/bky010.
[13] Feng Yongming. Study on Postmortem Distribution and Dynamic Distributions of Ethanol and Metabolites thereof in Rabbits[D]. Taiyuan: Shanxi Medical University, 2018.
[14] Feng Y M. Study on the postmortem distribution and dynamic distribution of ethanol and its metabolites in rabbits[D]. Taiyuan:ShanxiMedical University, 2018.
[15] Yu Mingjun, Chen Jiahui, Yang Xiuxiu, et al. Research of Generation of Ethanol, Ethyl Glucuronide (EtG), and Ethyl Sulfate (EtS) in Postmortem Blood Sample[J]. Chinese Journal of Forensic Medicine, 2020, 35 (02): 158-166. DOI: 10.13618/j.issn.1001-5728. 2020.02.011.
[16] Yu M J, Chen J H, Yang X X, et al. Research of generation of ethanol, ethyl glucuronide and ethyl sulfate in post-mortem blood sample[J].Chin J Forensic Med. 2020, 35(02):158-166.DOI:10.13618/j.issn.1001-5728.2020.02.011.
[17] Food and drug administration. The 2018 revised guidance: Bioanalytical method validation [S]. U.S.: Center for drug evaluation and research, 2018.
[18] Liu Xiaoxia. Study on Postmortem Redistribution of Ethanol in Dogs and Stability of Ethanol in Preserved Human Blood[D]. Taiyuan: Shanxi Medical University, 2007.
[19] Liu X X. The studys of the alcohol postmortem redistribution indogs and the stability in preservative samples[D]. Taiyuan:Shanxi Medical University, 2007.
[20] Wang H, Li B, Wang F, et al. Determination of Ethyl Glucuronide and Ethyl Sulfate in Human Whole Blood and Vitreous Humor by LC-MS-MS and Applications to the Interpretation of Postmortem Ethanol Findings[J]. J AnalToxicol. 2021; 45(5):484-489. doi:10.1093/jat/bkz082.
[21] Yun Keming. Forensic Toxicokinetics[M]. Beijing: People's Medical Publishing House, 2015: 935-936.
[22] Yun K M. Forensic Toxicokinetics[M]. Beijing: People's Medical Publishing House, 2015:935-936.
[23] Uno Y, Uehara S, Murayama N, et al. Expression of functional sulfotransferases (SULT) 1A1, 1A3, 1B1, 1C2, 1E1, and 2A1 in common marmosets[J]. Biochem Pharmacol. 2020, 180:114189. doi: 10.1016/j.bcp.2020.114189.
[24] Dhuria N V, Haro B, Kapadia A, et al. Recent developments inpredicting CYP-independent metabolism[J]. Drug Metab Rev. 2021, 53(2):188-206.doi: 10.1080/03602532.2021.1923728.
[25] King C D, Rios G R, Green M D, et al. UDP-glucuronosyltransferases[J]. Curr Drug Metab. 2000, 1(2):143-61. doi: 10.2174/1389200003339171.
[26] Stachel N, Skopp G. Identification and characterization of sulfonyltransferases catalyzing ethyl sulfate formation and their inhibition by polyphenols[J]. Int J Legal Med. 2016,130(1):139-46. doi: 10.1007/s00414-015-1159-5.
[27] Riley E, Bolton-Grob R, Liyou N, et al. Isolation and characterisation of a novel rabbit sulfotransferase isoform belonging to the SULT1A subfamily[J]. Int J Biochem Cell Biol. 2002, 34(8):958-69. doi:10.1016/s1357-2725(02)00017-1.
[28] Ren J, Barton C D, Sorenson K E, et al. Identification of a novel glucuronyl transferase from Streptomyces chromofuscus ATCC 4998₂ for natural product glucuronidation. Appl Microbiol Biotechnol. 2022,106(3):1165-1183.doi: 10.1007/s00253-022-11789-2.

What is claimed is:

1. Identification markers for the accurately distinguishing between antemortem alcohol consumption and postmortem alcohol introduction, comprising: a concentration ratio of ethanol in a heart blood sample in a dead body to ethanol in a vitreous humor sample in the dead body, concentrations of ethanol in a muscle of a lower limb and a testis in the dead body, and concentrations of EtG and EtS in the muscle of the lower limb and the testis in the dead body.

2. A method for accurately distinguishing between the antemortem alcohol consumption and the postmortem alcohol introduction using the identification markers according to claim 1, wherein
if the concentration ratio of the ethanol in the heart blood sample to the ethanol in the vitreous humor sample is less than 1 in the dead body, the antemortem alcohol consumption is determined for the dead body;
if the concentration ratio of the ethanol in the heart blood sample to the ethanol in the vitreous humor sample is more than or equal to 1, and ethanol shows positive in the muscle of the lower limb and the testis in the dead body, the antemortem alcohol consumption is determined for the dead body;
if the ethanol shows negative in the muscle of the lower limb and the testis, and EtG and EtS show positive in the muscle of the lower limb and the testis in the dead body, the antemortem alcohol consumption is determined for the dead body; and
if the concentration ratio of the ethanol in the heart blood sample to the ethanol in the vitreous humor sample is more than or equal to 1, the ethanol shows negative in the muscle of the lower limb and the testis, and the EtG and the EtS show negative in the muscle of the lower limb and the testis in the dead body, the postmortem alcohol introduction is determined for the dead body.
